(19) United States
(12) Patent Application Publication
(10) Pub. No.: US 2008/0065200 A1
(43) Pub. Date: Mar. 13, 2008

(54) BIFURCATED PROSTHESES HAVING DIFFERENTIAL DRUG COATINGS

(75) Inventors: Gary Binyamin, Palo Alto, CA (US); Eitan Konstantino, Orinda, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: TriReme Medical, Inc., Pleasanton, CA (US)

(21) Appl. No.: 11/851,643

(22) Filed: Sep. 7, 2007

Related U.S. Application Data
(60) Provisional application No. 60/824,884, filed on Sep. 7, 2006.

(51) Int. Cl. A61F 2/06

(52) U.S. Cl. 623/1.35

(57) ABSTRACT

Bifurcated stent includes a main stent body and a crown adapted to deploy it laterally into a branch vessel from the main stent body. A therapeutic agent or other material is coated on at least a portion of the crown of the stent, usually over the base of the crown adjacent to the main stent body. The main stent body is either free from coating or is coated in a different manner than is the crown portion of the stent.

### CROSS-REFERENCES TO RELATED APPLICATIONS

This application claims the benefit of prior provisional application 60/824,884 (Attorney Docket No.: 022246-000700US), filed on Sep. 7, 2006, the full disclosure of which is incorporated herein by reference.

### BACKGROUND OF THE INVENTION

1. Field of the Invention

This invention relates generally to the coating of medical devices for improved performance and biocompatibility. The invention focuses on coatings and application methods for use in cardiovascular devices, specifically to prevent restenosis in bifurcated for practically every material implanted into the body, excess response can lead to clinical complications or malfunction of the device. The interfacial properties of a material are responsible for the extent of elicited response. These include several characteristics including the surface chemistry, morphology, and synthetic history.

Coronary stents introduced in the late 1980s solved major complications associated with percutaneous transluminal coronary angioplasty (PTCA). Most notably, the risk of restenosis was reduced from 50-60% of treated lesions to 10-40%. Further observation around the mechanism of restenosis and intimal repair led to the introduction of drug eluting stents (DES) which has further reduced restenosis rates to 4-5%. Although long-term follow-up from the initial DES studies are not yet available, market acceptance in the US is estimated to be 90% of all stents placed in 2006.

Complicated lesions are a subset of all coronary lesions that are not well addressed by current stent technologies. Examples of these lesions include small vessels (less than 3 mm), bifurcations, long lesions, and left main disease. Of these bifurcated lesions are probably the largest subset, with approximately one third of all stented lesions being at bifurcations. These bifurcated lesions are difficult to access and treat with current technologies. In addition to being complicated procedural cases, they suffer from restenosis rates of over 20%, even when DES are used.

More recently there have been reports that the potential for late-thrombosis is higher for DES, leading to increased risk of acute myocardial infarction and other major acute coronary events (MACE) including death in patients. These results are attributed lack of endothelialization over the DES exacerbated by non-compliance with anti-coagulation medications (clopidogrel). The lack of healing in these stents and increased costs due to long-term clopidogrel treatment has prompted several institutions to re-evaluate the indiscriminate use of DES. Alternatives to DES in development include biodegradable drug eluting coatings, biodegradable stents, and alternative coatings. The alternative coatings have been suggested as a compromise between bare metal stents (BMS) and DES. They are often comprised of thin inorganic layers deposited on the stent surface and are beginning to show improved restenosis rates over BMS in preclinical and initial clinical studies.

For these reasons, it would be desirable to provide drug eluding bifurcated stents specifically designed and intended for placement at bifurcations in the vasculature. It would be particularly desirable if the bifurcated stent designs would permit the drugs to be released into targeted portions of the bifurcation to increase effectiveness, preferably while decreasing the risk of late thrombosis. At least some of these objections will be met by the inventions described below.

2. Description of the Background Art

The following publications describe the placement of drug eluding stents in both normal and bifurcated vasculature: Anis, R R; Karsch, K R. The future of drug eluting stents. Heart. (2006); 92: 585-588; Erne, P; Schier, M; Resink, T J. The Road to Bioabsorbable Stents: Reaching Clinical Reality? Cardiovasc Intervent Radiol (2006); 29: 11-16; Medtech insight Interventional Cardiology Report. May 2006: 169-180; Tung, R; Kaul, S; Diamond, G A; Shah, P K. Drug-Eluting Stents for the Management of Restenosis: A Critical Appraisal of the Evidence. Anal Intern Med. (2006); 144(12): 913-919; Feres, F; Costa, J R; Abizaid, A. Very Late Thrombosis After Drug-Eluting Stents. Cath. Cardiovasc. Interven. 2006; 63: 83-88; Westphal S P. Concerns Prompt Some Hospitals to Pare Use of Drug-Coated Stents; Wall St. Journal. 2006: A1; Wieneke, J; Sawitowski, T; Wnendt, S; Fischer, A; Dirsch, O; Karoussos, I A; Erbel, R. Stent Coating: A New Approach in Interventional Cardiology. Herz. 2002; 27(6): 518-526; and Gunn, J; Cumberland, D. Stent Coatings and Local Drug Delivery. Eur. Heart J. 1999; 20: 1693-1700.

### BRIEF SUMMARY OF THE INVENTION

This invention is for improved coatings and application of coatings for medical devices, specifically to minimize or prevent restenosis in bifurcated stents. While examples and detail will be provided to aid in the enablement of the coating, these should not limit the disclosure. Any suitable systems, methods, or materials that function in similar ways, as known to a practitioner skilled in the art, may alternatively be used and should be considered within the scope of this disclosure. In particular, the invention provides for vascular stents and other prosetheses which are “differentially” coated at a region which will be located near a bifurcation. Such differential coating may comprise coating with an anti-hyperplasia drug over a portion of the stent near the bifurcation with no coating, or a different coating, over remaining portions of the stent.

In a first aspect of the present invention, a bifurcated stent or other prosthesis comprises a main stent body adapted for placement in a body lumen adjacent to a branch lumen. A crown on the main stent body is adapted to be deployed laterally outwardly from the stent body and into the branch lumen. A coating is provided over at least a portion of the stent, where the coating includes at least a first coating region over at least a portion of the crown which differs from a second coating region over the main stent body. For example, the coating over the crown of the stent may be formed over all or a portion of the stent, typically being disposed primarily or solely over the base region of the stent adjacent to the main body of the stent. The coating region over the crown may comprise any one of a variety of materials, particularly including anti-hyperplasia or anti-neoplastic therapeutic agents, as described in more detail below. The coating may comprise two or more of such agents and the agent(s) may be deposited at a uniform or non-uniform concentration over the crown.

The second coating region over the main stent body will differ in some therapeutic, chemical, or physical aspect relative to the coating region over the crown. In many cases, the main stent body may be free or substantially free from any coating, or the concentration or nature of the coating may differ from that disposed over the crown. For example, the main stent body may be coated with one or more therapeutic agents, but the coating may be in different concentrations or may be of different materials. Alternatively, the coatings may comprise polymeric or other carriers which provide different release rates than found in the first coating region on the crown. In general, the present invention allows the crown and main stent body to be differentially coated in order to permit different treatment of the blood vessels in regions at or adjacent to the side branch from regions more remote from the side branch.

The bifurcated stents according to the present invention may be balloon-expandable, and in the specific embodiments may comprise a stent having a crown which deploys in response to balloon expansion of the main stent body, as described in co-pending application Ser. No. 11/330,382, filed on Jan. 10, 2006, now the full disclosure of which is incorporated herein by reference. In this specific example, the crown may comprise a plurality of elements which are connected to the main body so that expansion of the main body applies an opening force to the elements to cause the elements to move laterally away from an outer surface of the main body. The present invention is not limited to such balloon-expandable stents and will include self-expanding stent structures which comprise both the main stent body and a laterally deployable crown.

The coatings of the present invention include any and all known and yet to be developed methods for sequestering therapeutic and other agents on or within the stent structure. Most typically, the coating will comprise one or more layers of a therapeutic agent deposited over an outer surface of at least a portion of the main stent and/or crown, typically being disposed in a polymeric or other carrier. Such conventional coatings may be applied by spraying, vapor deposition, dipping, painting, ink jet coating, aerosol coating, contact printing, stamp printing, physical vapor deposition, chemical vapor deposition, electroless deposition, electrolytic deposition, sol-gel deposition, and sputtering. To achieve desired coating patterns, the stent surfaces or a portion thereof may be masked using conventional photolithographic or other techniques. Alternatively, coatings could be applied uniformly over the stent surfaces and thereafter modified, for example by etching, washing, or otherwise removing or modifying portions of the coating to achieve the differential coatings of the present invention.

As an alternative to such conventional coating techniques, the coating may comprise a reservoir which holds a therapeutic agent. For example, the stents may include one or more wells formed in the surface for sequestering the agent. Alternatively, at least a portion of the stent may have a porous surface, or may include a sintered structure which allows for the absorption or adsorption of the therapeutic or other agent. Additionally, the stents may comprise porous or non-porous barriers formed over the coatings, reservoirs, porous structures, or the like in order to further modify the release characteristics of the therapeutic or other agents.

In a second aspect, the present invention provides methods for delivering a therapeutic agent to a bifurcation in a body lumen, typically in the vasculature, and more particularly in the coronary or peripheral vasculature. The methods comprise advancing a stent into a body lumen and deploying a crown of the stent into a side branch of the lumen while a main body of the stent remains in the body lumen. A first therapeutic agent formulation is present on the crown, and the main body has a second therapeutic agent formulation (which differs from the first in composition, concentration, release characteristics, or the like) or no therapeutic agent whatsoever. The first therapeutic agent will typically comprise an anti-hyperplasia or anti-proliferative drug and the main body will preferably be substantially free from therapeutic agent. In a specific embodiment, the therapeutic agent is concentrated about the base of the crown so that agent is delivered to an os region of the side branch to achieve a concentrated therapeutic effect. In some embodiments, the main body will be at least partially coated with a second therapeutic agent which has a different activity or concentration than that of the first therapeutic agent.

### BRIEF DESCRIPTION OF THE DRAWINGS

FIG. 1 illustrates a bifurcated stent having a main stent body and a laterally deployable crown, of the type which may be coated in accordance with the principles of the present invention.

FIGS. 2 through 6 illustrate the stent of FIG. 1 showing different regions of the stent which may be coated in accordance with the principles of the present invention.

FIGS. 7 through 11 illustrate different techniques for coating stents and stent surfaces.

FIGS. 12A through 12D illustrate deployment of a bifurcated stent coated with an anti-hyperplasia agent around the base of a crown extending into a branch lumen in the vasculature.

### DETAILED DESCRIPTION OF THE INVENTION

This invention comprises coatings and application of coatings for medical devices, specifically to minimize or prevent restenosis in bifurcated stents. While examples and detail will be provided to aid in the enablement of the coating, these should not limit the disclosure. Any suitable systems, methods, or materials that function in similar ways, as known to a practitioner skilled in the art, may alternatively be used and should be considered within the scope of this disclosure.

The coatings may be applied to a coronary bifurcated stent produced out of austenitic stainless steel (type 316L). This embodiment should not limit this invention in the type of substrate material or the design of the device to which the coating is applied. Other regions in which bifurcated stents might be applicable include but are not limited to the peripheral vascular system including the biliary tree, neurovasculature, and the tracheo-bronchial system.

The term coating and layer are used interchangeably in this document. The abluminal side of the stent refers to the outer most layer of the device, that faces the vessel when implanted. The luminal side of the stent refers to the inner most layer of the device that faces the blood flow when implanted. The crown refers to the portion of the bifurcated stent that emerges from the axial plane to reside in the side branch upon deployment.

In a preferred embodiment the bifurcated stent is coated with a single layer using methods known to the art. This layer would preferentially be a polymer containing one or more therapeutic molecules, specifically a durable polymer containing an anti-proliferative. The coating can provide homogenous or inhomogenous coverage of the stent, including micro- and nano-structural units. For therapeutics contained in these layers, elution profiles and dosing can be modified to achieve desired properties. It is possible that therapeutic and loading parameters could be patient specific. The coating can cover only the structural elements of the stent or reside in-between structural elements of the stent.

Polymers used for drug eluting substrates include both durable and non-durable (biodegradable) materials. Degradable polymers include, but are not limited to, poly-L-lactic acid, polyglycolic acid, poly(D,L-lactide/glycolide), polycaprolactone, polyorthoesters, polyanhidrides, poly(hydroxybutyrae-hydroxyvalerate), tyrosine derived carbonate, polyethylene oxide, polybutyleneterphthalate, polyphosphazines, polypropylene fumarate, polyhydroxyalkanoates, polyanhydrides, polyamino acid, polysaccharide, and co-polymers including these. Alternatively, a polymer that degrades in response to a stimulus such as an enzyme or energy application is considered degradable. Durable polymers include, but are not limited to, silicones, phosphorylcholine, polyurethane, polyehtyleneterphthalate, polymethylmethacrylate, poly(ethylmethacrylate/n-butylmethacrylate), parylene C, polyethylene-co-vinyl acetate, polyfluoroalkoxyphasphazine, poly(styrene-b-isobutylene-b-styrene), poly-butyl methacrylate, poly-butadiene, and co-polymers containing these.

Therapeutics classes considered for this application include, but are not limited to, small molecules, cell therapy, biologic molecules, polymers, genes, plasmids, proteins, peptides, RNAi, antibodies, and growth factors. Small molecule therapeutics used for drug eluting stents include, but are not limited to, sirolimus, tacrolimus, everolimus, microphenolic acid, ABT-578, biolimus, taxol, tyxane, QP2, dexamethasone, 17-beta-estradiol, batimastat, actinomycin D, methotrexate, angiopeptin, tyrosine kinase inhibitors, vincristine, mitomycin, cyclosporine, Mg, platelet derived growth factor (PDGF), tyrophostin AGL-2043, heparin, statins (lovastatin, pravastatin, simvastatin, etc.), structural analogs of the these compounds, and pro-drugs of these compounds. For therapeutics contained in polymer layers, elution profiles and dosing can be modified to achieve desired properties. It is possible that therapeutic and loading parameters could be patient specific. Cell therapies include methods for pre-endothelialization prior to implantation. Methods and treatments for promoting in vivo endothelialization are preferred.

Methods of deposition include, but are not limited to, dip coating, spray coating, ink jet coating, aerosol coating, contact printing, stamp printing, physical-vapor deposition, chemical vapor deposition, electroless deposition, electrolytic deposition, sol-gel deposition, and sputtering. This may or may not require a masking step. Specifically deposition of a layer only at the crown could entail the development of contact printing or stamping, having a fixture that contacts the stent surface but only transfers material at the area of the crown. Computer controlled ink jet printing for exact tolerance coatings applied at the crown is also possible. Alternatively a post-process step to activate the coating selectively could be needed. The application of light at a specific wavelength, voltage, current, thermal curing, solvent evaporation, or formation of electrostatic adducts are all possible activation methods.

In another preferred embodiment the bifurcated stent is coated with a single layer using methods known to the art. This layer would preferentially be an alternative layer to polymers. Specifically this alternative layer could be inorganic such as metal, ceramic, oxide, micro-, or nano-structural units. Conversely the alternative layer could be an organic such as an oil, emulsion, porous layer, micro-, or nano-structural units. This layer can include one or more therapeutic molecules, but it is not necessary for performance of the layer. The coating can provide homogenous or inhomogenous coverage of the stent. The coating can cover only the structural elements of the stents or reside in-between structural elements of the stent.

Alternative coatings can be composed of, but are not limited to, titanium nitric oxide, gold, titanium nitride, iridium, ruthenium, osmium, rhodium, palladium, platinum, chromium, aluminum, tantalum, titanium, zirconium, niobium, molybdenum, silver, antimony, tellurium, iodine, barium, lanthanum, hafnium, rhenium, platinum, silicon carbide, carbon, tungsten, indium, tin, indium tin oxide, hydroxyapatite, oxides of pure materials, combination of these materials, or oxides of combinations of these materials. Also included are biodegradable metals including, but not limited to, iron, magnesium, and alloys containing these materials. Inorganic-organic hybrid materials are also included; examples include the sol-gel formation of silica from silanes doped with organic modifiers such as poly(tetramethylene oxide), polycapralactone, hydroxyethyl methacrylate, polyethylene glycol, polypropylene oxide, polyvinylpyrrolidone, hydroxpropyl cellulose, polyurethanes, and polystyrene sulfonic acid. Layers can be homogenous or inhomogenous. Layers can contain a single material, multiple materials in discrete zones, or mixtures of materials. There can be a single layer of these materials, multiple layers of variable thicknesses, or nanostructural elements on the surface. Coating layers can be in any of several forms including but not limited to amorphous oxide layers, single crystal oxide layers, and polycrystalline oxide layers. One or more therapeutics may be included in these alternative layers, but are not required for performance of the layer. For therapeutics contained in alternative layers, elution profiles and dosing can be modified to achieve desired properties. It is possible that therapeutic and loading parameters could be patient specific. These layers can be of variable porosity. These could be made of zeolite-like materials, aerogels, or xerogels.

One specific embodiment would be the coating of a bifurcated stent produced from stainless steel with a layer of osmium. With the use of the OPC Osmium Plasma Coaters (Nippon Laser and Electronics Laboratory, Japan) a thin film of osmium (on the order of 10 nm-10 μm thick) is introduced homogenously over the entire surface of the stent. Alternatively, a similar layer could be formed on the stent by dip coating in an organic precursor with a subsequent treatment step (eg. heating) to form an inorganic layer.

In a preferred embodiment the bifurcated stent is coated with multiple layers using methods known in the art. The layers would include two or more layers of polymer containing therapeutic, inorganic layer with or without therapeutic, or a combination therein. The coating can provide homogenous or inhomogenous coverage of the stent. The coating can cover only the structural elements of the stents or reside in-between structural elements of the stent.

Another specific embodiment would be the coating of a bifurcated stent produced from stainless steel. The struts of the stent are pre-coated with a layer of parylene C. Two durable polymers, polyethylene-co-vinyl acetate and poly n-butyl methacrylate, are combined in a preferred ratio of 2:1. These are then applied to the parylene C coated stent to form a durable polymer layer. An additional layer of poly n-butyl methacrylate can also be applied atop the layers for the purpose of modifying subsequent transport properties out the layers. Alternatively the durable polymers applied to the struts of the stent could be poly(styrene-b-isobutylene-b-styrene) or a combination of poly-butyl methacrylate and poly-butadiene. A therapeutic could be included in the durable polymer layer during the coating process, preferentially a hydrophobic small molecule such as Sirolimus.

In a preferred embodiment the coating layer is or contains a radiopaque material (eg. Zr, Nb, Mo, Tc, Ru, Rh, Pd, Ag, Cd, In, Sn, Sb, Te, I, Ba, La, Hf, Ta, W, Re, Os, Ir, Pt, and Au).

In another preferred embodiment, the stent itself has micropores created on the surface of the stent. The pores are subsequently filled with a therapeutic or combination of layer and therapeutic.

In a preferred embodiment, titanium metal is deposited on the bifurcated stent. Titanium can be electrodeposited by using a two or three electrode system in a deposition solution. (Natarajan C.; Nogomi G. Cathodic Electrodeposition of Nanocrystalline Titanium Dioxide Thin Films. JECS 1996; 143(5): 1547-1550.) In this case the bifurcated stent would be the working electrode, platinum wire would be the counter electrode, and an appropriate reference electrode used. The deposition solution would be composed of titanium powder dissolved in a hydrogen peroxide and ammonia solution, allowed to mix under application of heat until a yellow gel is obtained. The solution would be dissolved in an acidic solution, 2M sulfuric acid or nitric acid, and added to an aqueous potassium nitrate solution at a concentration of 5-500 mM. The pH would then be adjusted to be between 1-3 using ammonia and nitric acid. Deposition would occur potentiostatically between −0.9 to −1.4V (versus SCE). Formation of the oxide layer could then be achieved, preferentially by heating.

In another preferred embodiment, titanium metal is deposited on the bifurcated stent. Titanium can be deposited electrolessly by immersion into a deposition solution and allowing it to cure. Shimizu K; Imai H; Hirashima H; Tsukuma K. Low-temperature synthesis of anatase thin films on glass and organic substrates by direct deposition from aqueous solutions. Thin Solid Films 1999; 351: 220-224.) To 50 mL of deionized water, the pH adjusted to (1.0-3.1) using HCl or NH₄OH. TiF₄ is dissolved in the solution to yield a concentration of 0.03-0.1 M and maintained at a temperature of 40°-70° C. The stent is immersed into the solution for 0.5-10 hours

In a preferred embodiment, magnesium metal is deposited on the bifurcated stent. Magnesium can be electrodeposited by using a two or three electrode system in a deposition solution. (Pawar S H; Jadhav A B; Shirage P M; Shivagan D D. Electrochemical synthesis of superconducting MgB2 thin films: a novel potential technique.) The stent would be the working electrode, graphite would be the counter electrode, and a reference electrode used. The deposition solution would be composed of 50 mM magnesium acetate dissolved in water or dimethyl sulphoxide (DMSO). Deposition would occur potentiostatically at −2.36V (versus NHE) for aqueous or −1.1 to −2.36 (versus NHE) for DMSO.

In a preferred embodiment, the stent in its entirety is coated with one type of coating and only the crown of the stent is then coated with a second type of coating (differential coating). The initial coating could be composed of an alternative coating. The purpose of this alternative coating could be to promote healing, minimize inflammation, and prevent thrombosis. The second coating just on the crown could be composed of an anti-restenotic layer that could elute an anti-proliferative therapeutic. The coating on the crown could be a homogenous coating on the crown elements, an inhomogenous coating on specific areas of the crown, or a coating just on the marker elements that reside within the crown. Alternatively, upon deployment of the crown into the side branch vessel enough stress is placed upon the coating to promote increase in pore size or crack formation and propagation within the coating resulting in initiation and promotion of therapeutic elution. Still further alternatively, the crown could be coated while remaining portions of the stent remain uncoated.

In a preferred embodiment, the main body of the stent that resides in the main vessel is coated with one type of coating and the crown of the stent that resides in the side branch is coated with a second type of coating. The coating on the main stent could be composed of an alternative coating. The purpose of this alternative coating could be to promote healing, minimize inflammation, and prevent thrombosis. The coating on the crown could be composed of an anti-restenotic layer that could elute an anti-proliferative therapeutic. The coating on the crown could be a homogenous coating on the crown elements, an inhomogenous coating on specific areas of the crown, or a coating just on the marker elements that reside within the crown. Alternatively, upon deployment of the crown into the side branch vessel enough stress is placed upon the coating to promote increase in pore size or crack formation and propagation within the coating resulting in initiation and promotion of therapeutic elution.

In a preferred embodiment, the marker elements in the crown may serve multiple purposes. These include but not limited to being radiopaque, containing a therapeutic, promoting endothelialization, minimizing restenosis, and sensing applications. The marker elements could be composed of radiopaque materials, coated with radiopaque materials, or have radiopaque materials incorporated into a structure made of non-radiopaque materials. Examples of radiopaque materials include markers, films, and particles. These could be metals, polymers, or other radiopaque materials. Alternatively the crown or the markers could be composed of materials, coated with materials, or incorporate materials that respond to MR such as ferromagnetic materials. These could be used for MR visualization or for therapeutic application. The induction of local heating through the use of magnetic fields on a material can be used for therapeutic or visualization purposes. Alternatively the crown or markers could be composed of materials, coated with materials, or incorporate materials that respond to other energy sources such as IR or near-IR light. These could be used for visualization or for therapeutic application. The induction of local heating through the use of light application on a material can be used for therapeutic or visualization purposes. Alternatively the crown or the markers could be composed of materials, coated with materials, or incorporate materials that respond to ultrasound by altering the propagation rate of the sound waves, such as gas-filled nanoparticles. These could be used for ultrasound visualization (eg. IVUS) or for therapeutic application (eg. cavitation induced release of drugs, cavitation induced heating). Alternatively the markers in the crown could be composed of materials that elute one or more therapeutics. These materials could be durable polymers, biodegradable polymers, or alternative coatings. Therapeutics can include any of those previously mentioned. In a preferred embodiment the marker elements can be micro (nano) electricalmechanical systems (MEMS and NEMS) sensors or RFID tags for identification and transmission of information. These can be used for visualization purposes to aid in placement and localization. Alternatively, integrated sensors can be used post-placement to monitor patient information such as pressure, strain, or flow. Such measurements can be used to track cardiac performance, track possible restenosis, and monitor for myocardial infarction These would be used in combination with external telemetry elements for the collection of data, methods of disseminating information to appropriate storage systems, and mechanisms through which clinical evaluation of these data are performed.

In a preferred embodiment, the abluminal surface of the stent is coated with one type of coating and the luminal surface of the stent is coated with a second type of coating. The coating on the abluminal surface could be composed in such a way to promote healing or endothelialization. The coating on the luminal surface could be composed in such a way to be anti-thrombogenic or anti-proliferative.

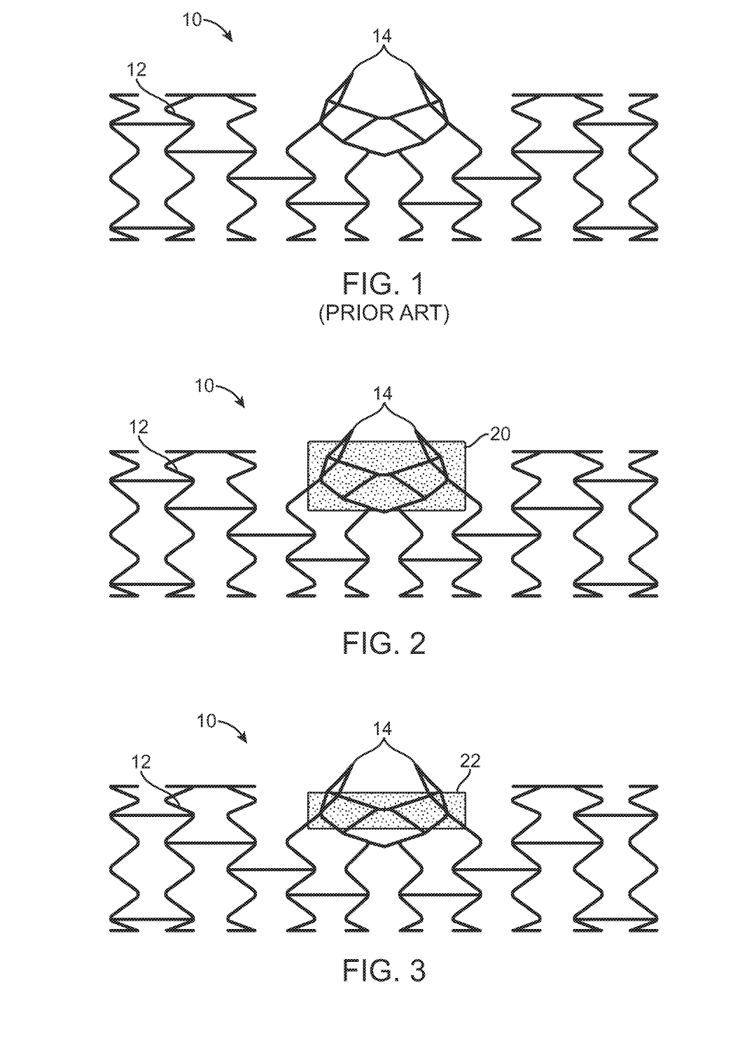

Referring now to FIG. 1, a bifurcated stent 10 having a main body 12 and a crown 14 for deployment in a side branch lumen is illustrated. Stent 14 has a self-opening crown 14 as generally described in co-pending, commonly owned application Ser. No. 11/330,382, the disclosure of which has been previously incorporated herein by reference. It will be appreciated that the present invention is not limited to such self-deploying crowns, but will apply to any bifurcated stent which includes both a main stent body and structure, referred to herein as a crown, which opens into a side branch lumen through an os in a main vessel.

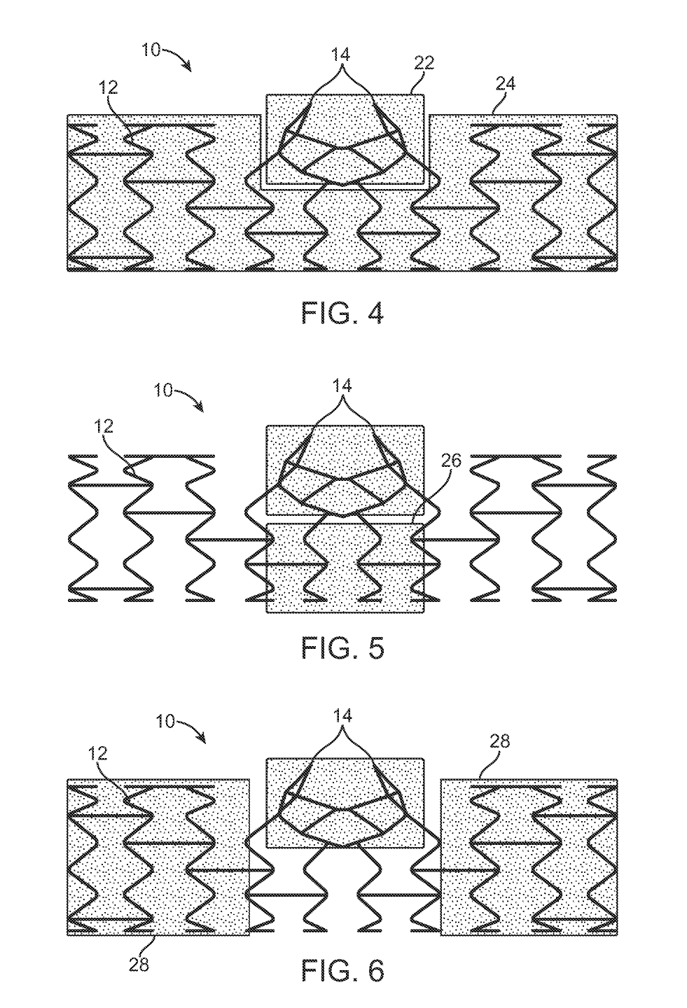

In accordance with the present invention, at least a portion of the crown region 20, as shaded in FIG. 2, will be coated in any of the ways described above. Typically, the coating will be a therapeutically active coating, more typically being an anti-hyperplasia or anti-proliferative agent, such as those listed above. A crown may be covered in its entirety, as shown in FIG. 2, or may be covered only in a base region 22, as shown in FIG. 3. In addition to coating the crown of the stent, as shown in either FIG. 2 or 3, the main body may also be coated, as shown in the shaded region 24 in FIG. 4. The main body may be coated in its entirety, or may be only partially coated, as shown in region 26 in FIG. 5. Within the coated region, the coating may be varied, either in material, concentration, release characteristics, or otherwise as described above. Various patterns of coating the main body stent may be employed, such as coating only the ends of the stent in region 28, as shown in FIG. 6.

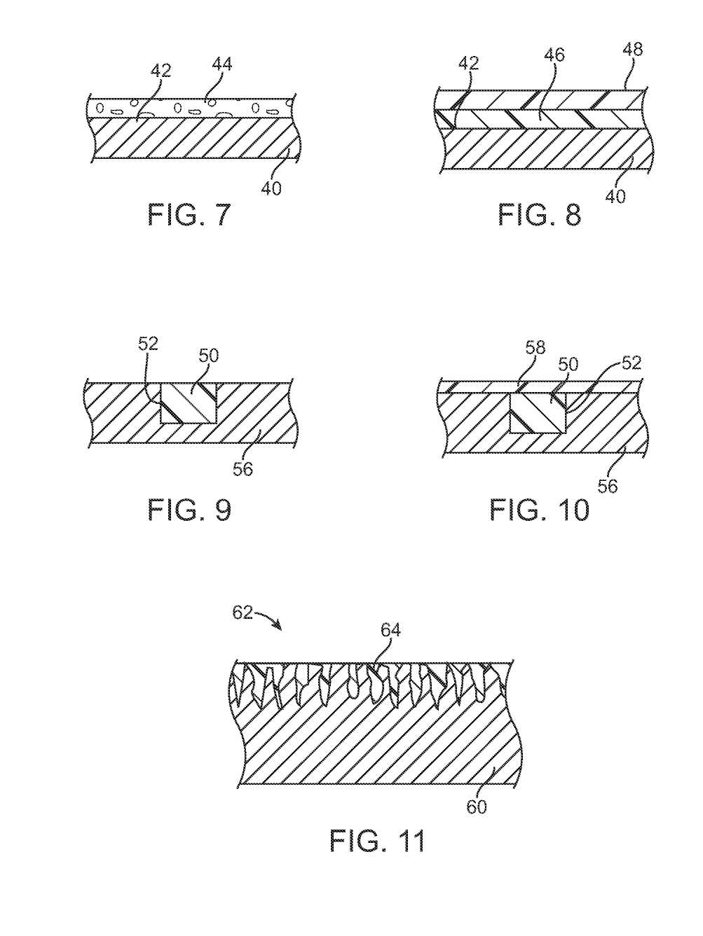

Various methods for coating the struts of the stent are illustrated in FIGS. 7 through 11. For example, a strut 40 may have an abluminal side 42 which is coated with a polymeric carrier 44 having a desired therapeutic or other agent disbursed therein. The agent may either be released through pores of the polymeric carrier 44 or may be released as the carrier erodes in the vascular or other luminal environment.

As shown in FIG. 8, the abluminal surface 42 of strut 40 may have at least two layers 46 and 48 applied thereto. The base layer 46 may be provided as an interface for the upper layer 48. Alternatively, both layers 46 and 48 may be active and/or may contain a desired therapeutic or other agent.

As shown in FIG. 9, a polymeric carrier or therapeutic agent 50 may be deposited in a well 52 or other reservoir formed in a strut 56. Optionally, as shown in FIG. 10, the well 52 may be covered with a membrane or layer 58 which controls release of therapeutic agent from the well The layer 58 may be porous in order to control the release rate of therapeutic agent from the well. Alternatively, the layer 58 may be non-porous in order to inhibit release of therapeutic or other agent until the layer is either degraded, cracked, or otherwise breached to permit release.

In a still further embodiment of the present invention, the coating may be deposited in a porous surface 62 of a strut 60. This structure may have a membrane formed over it, similar to layer 58 in FIG. 10, or may be directly exposed to the vascular or other luminal environment. The material 64 comprising the coating deposited in the pores may be a therapeutic agent, a polymeric or other carrier incorporating a therapeutic agent, or any other desired material.

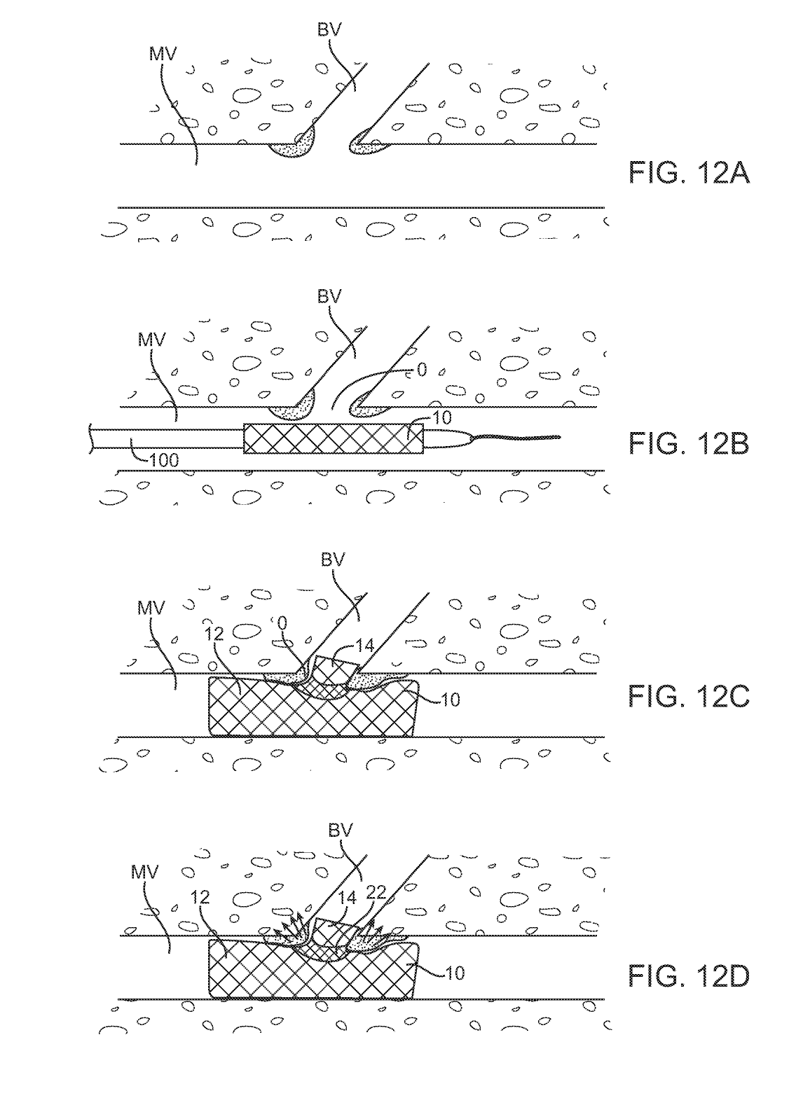

Referring now to FIGS. 12A through 12D, deployment of the bifurcated stent 10 having an anti-hyperplasia agent disposed about a base of crown 14, as illustrated in FIG. 3, will be described. A blood vessel, such as a coronary artery or a peripheral artery or vein, includes a main vessel MV and a branch vessel BV, as shown in FIG. 12A. A catheter 100 carrying stent 10 on a deployment balloon is advanced through the main vessel so that it is aligned with the os O to the branch vessel BV. After proper alignment of the crown portion is achieved (as described, for example, in co-pending, commonly owned application Ser. No. 11/439,707, the full disclosure of which is incorporated herein by reference), the main stent body 12 may be expanded and the crown portion 14 deployed into the branch vessel BV through the os O, as shown in FIG. 12C. After deployment, the coated region 22 of the base of the crown 14 releases anti-hyperplasia agent preferentially into the region of the os which is at high risk of hyperplasia and restenosis, as shown by the arrows in FIG. 12D.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A bifurcated stent comprising:
a main stent body adapted for placement in a body lumen adjacent to a branch lumen; and
a crown on the stent body adapted to be deployed laterally outwardly from the stent body and into the branch lumen;
a coating over at least a portion of the stent, wherein a coating region over the crown of the stent differs from a coating region over the main stent body.

2. A bifurcated stent as in claim 1, wherein the stent is balloon-expandable.

3. A bifurcated stent as in claim 2, wherein the crown deploys in response to balloon expansion of the main stent body.

4. A bifurcated stent as in claim 3, wherein the crown comprises a plurality of elements which are connected to the main body so that expansion of the main body applies an opening force to the elements to cause the elements to move laterally away from an outer surface of the main body.

5. A bifurcated stent as in claim 1, wherein the stent is self-expanding.

6. A bifurcated stent as in claim 1, wherein the coating comprises a layer of a therapeutic agent deposited over an outer surface of at least a portion of the main stent and/or crown.

7. A bifurcated stent as in claim 6, wherein the layer is applied by one or more of spraying, vapor deposition, dipping, painting, ink jet coating, aerosol coating, contact printing, stamp printing, physical vapor deposition, chemical vapor deposition, electroless deposition, electrolytic deposition, sol-gel deposition, and sputteringspraying, vapor deposition, dipping, or painting.

8. A bifurcated stent as in claim 1, wherein the coating comprises a reservoir which holds a therapeutic agent.

9. A bifurcated stent as in claim 8, wherein the reservoir comprises a well in a surface of the stent.

10. A bifurcated stent as in claim 8, wherein the reservoir comprises a porous or non-porous barrier formed over the therapeutic agent.

11. A bifurcated stent as in claim 1, wherein the coating over the entire crown is coated.

12. A bifurcated stent as in claim 1, wherein the entire main stent body is free from coating.

13. A bifurcated stent as in claim 1, wherein at least a portion of the main stent body is coated with a different material or the same material in a different amount.

14. A bifurcated stent as in claim 1, wherein only a base portion of the crown is coated.

15. A bifurcated stent as in claim 14, wherein the entire main stent body is free from coating.

16. A bifurcated stent as in claim 14, wherein at least a portion of the main stent body is coated with a different material or the same material in a different amount.

17. A method for delivering a therapeutic agent to a bifurcation in a body lumen, said method comprising:
advancing a stent into the body lumen; and
deploying a crown of the stent into a side branch of the lumen while a main body of the stent remains in the body lumen;
wherein a first therapeutic agent formulation is present on the crown and the main body has a second therapeutic agent formulation or no therapeutic agent.

18. A method as in claim 14, wherein the first therapeutic agent comprises an anti-proliferative drug and main body has no therapeutic agent.

19. A method as in claim 18, wherein the first therapeutic agent is concentrated about the base of the crown so that agent is delivered to an os of the side branch.

20. A method as in claim 18, wherein the main body is at least partially coated with a second therapeutic agent and the second therapeutic agent has a different activity or concentration than the first therapeutic agent.
